(19) United States
(12) Patent Application Publication
(10) Pub. No.: US 2003/0206955 A1
(43) Pub. Date: Nov. 6, 2003

(54) Nanoemulsions, compositions comprising such nanoemulsions, and methods of using such nanoemulsions

(75) Inventors: Odile Sonneville-Aubrun, Antony (FR); Jean-Thierry Simonnet, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, PARIS (FR)

(21) Appl. No.: 09/860,466

(22) Filed: May 21, 2001

(30) Foreign Application Priority Data
May 22, 2000 (FR) 0006511

(51) Int. Cl. A61K 9/14; A61K 7/00

(52) U.S. Cl. 424/486; 424/401

(57) ABSTRACT

The present application relates to an oil-in-water nanoemulsions which comprising an oily phase dispersed in an aqueous phase, the oil globules of which have a mean size of less than 100 nm, characterized in that it comprises at least one amphiphilic lipid chosen from nonionic amphiphilic lipids, anionic amphiphilic lipids and their mixtures and at least one anionic polymer which contains at least one hydrophobic chain and in which the weight ratio of the amount of oily phase to the amount of amphiphilic lipid ranges from 1.2 to 10. The anionic polymer can in particular be an acrylic or methacrylic acid copolymer or a 2-acrylamido-2-methylpropanesulphonic acid copolymer, in particular an acrylic or methacrylic acid terpolymer. The nanoemulsion obtained is transparent and stable on storage. It can constitute a composition for topical use and in particular for cosmetic or dermatological use, a pharmaceutical composition, or an ophthalmological composition.

### CROSS REFERENCES TO RELATED APPLICATIONS

This application claims priority to French Patent Application No. 0006511, filed on May 22, 2000, and which is incorporated herein by reference in its entirety.

### BACKGROUND OF THE INVENTION

1. Field of the Invention

The present invention relates to nanoemulsions comprising at least one nonionic and/or anionic amphiphilic lipid and at least one anionic polymer which comprises at least one hydrophobic chain. The present invention also relates to the use of the such nanoemulsions in topical application, in particular in the cosmetic and dermatological fields and in the pharmaceutical and/or ophthalmological fields.

2. Discussion of the Background

Oil-in-water (O/W) emulsions are well known in the field of cosmetics and dermatology, in particular for the preparation of cosmetic products, such as milks, creams, tonics, sera, or toilet waters.

Nanoemulsions are O/W emulsions in which the oily globules have a size of less than 100 nm, and the oily globules are stabilized by a wreath of amphiphilic lipids, which can optionally form a liquid crystal phase of the lamellar type, situated at the oil/aqueous phase interface. The transparency of these emulsions originates from the small size of the oily globules, a small size obtained by virtue of the use of mechanical energy and in particular of a high-pressure homogenizer. Nanoemulsions are to be distinguished from microemulsions by their structure. This is because microemulsions are thermodynamically stable dispersions composed of amphiphilic lipid micelles which are swollen by oil. Furthermore, microemulsions do not require high mechanical energy to be prepared; they are formed spontaneously by simply bringing the constituents into contact. The major disadvantages of microemulsions are related to their high proportion of surfactants, resulting in intolerance and leading to a sticky feel when applied to the skin. Furthermore, their formulation range is generally very narrow, and their temperature stability is very limited.

Nanoemulsions comprise one or more amphiphilic lipid(s). The term “amphiphilic lipid” is understood to mean here any molecule having a bipolar structure, that is to say comprising at least one hydrophobic part and at least one hydrophilic part, and having the property of reducing the surface tension of water (γ<55 mN/m) and of reducing the interfacial tension between water and an oily phase. Synonyms for amphiphilic lipid are, for example: surfactant, surface-active agent, emulsifier.

Published European patent applications EP-A-728 460 and EP-A-780 114 disclose nanoemulsions based on liquid nonionic amphiphilic lipids or on silicone surfactants. Nanoemulsions are also disclosed in published French patent applications FR-A-2 787 026, FR-A-2 787 027, FR-A-2 787 325, FR-A-2 787 326, FR-A-2 787 703 and FR-A-2 787 728 and in French patent applications FR 2 788 007, FR 2 788 449, and FR 2 788 980.

In order for the nanoemulsions as disclosed in these documents to be able to be used as milks or creams and in particular in the care field, they have to be rendered thicker, and, thus, their viscosity has to be increased. The are two means for increasing the viscosity of a nanoemulsion. The first means consists of increasing the fraction of the dispersed oily phase. This is because it is generally found that, from 22% by weight of oily phase with respect to the total weight of the composition, the viscosity increases as a function of the level of oil. This method, disclosed in the above-mentioned applications, makes it possible to obtain thick, transparent and stable compositions. However, the constraint of such a method for thickening nanoemulsions is the need to have a high level of oil, which is not always desired as the formulas obtained are richer (high level of fatty phase) and the viscosity range is narrower.

The second means for increasing the viscosity of a nanoemulsion consists of adding a hydrophilic polymer to the nanoemulsion, which polymer, by gelling of the continuous aqueous phase, will increase the viscosity of the combined mixture, even with low levels of oil. In the abovementioned applications, the addition of hydrophilic polymers, such as cellulose derivatives, algal derivatives, natural gums and synthetic polymers, such as polymers and copolymers of carboxyvinyl acids, for example Carbopols is envisaged. Unfortunately, the products obtained are not always transparent or not always transparent under conditions of low polymer concentration, which limits their effect.

Thus, there remains a need for a thickening system which makes it possible to suitably thicken a composition in the form of an oil-in-water nanoemulsion without influencing the cosmetic properties of the composition, in particular without influencing the transparent nature of the nanoemulsion, whatever the level of oil which it is desired to use.

### SUMMARY OF THE INVENTION

Accordingly, it is one object of the present invention to provide novel nanoemulsions.

It is another object of the present invention to provide novel nanoemulsions which are suitable for use as a milk or cream.

It is another object of the present invention to provide novel compositions which contain such a nanoemulsion.

It is another object of the present invention to provide novel pharmaceutical compositions which contain such a nanoemulsion.

It is another object of the present invention to provide novel ophthalmic compositions which contain such a nanoemulsion.

It is another object of the present invention to provide novel cosmetic or dermatological compositions which contain such a nanoemulsion.

It is another object of the present invention to provide novel methods for preparing such a nanoemulsion.

It is another object of the present invention to provide novel methods of treating the skin, mucous membranes, scalp, and/or hair with a composition which contains such a nanoemulsion.

It is another object of the present invention to provide novel methods of making up the skin with a composition which contains such a nanoemulsion.

These and other objects, which will become apparent during the following detailed description, have been achieved by the inventors' unexpected discovery that it is possible to thicken nanoemulsions with anionic polymers, preferably water-soluble or water-dispersible anionic polymers, comprising at least one hydrophobic chain.

Thus, in a first embodiment, the present invention provides oil-in-water nanoemulsions, comprising oily phase globules dispersed in an aqueous phase, wherein said oily phase globules have an average size of less than 100 nm, and wherein said nanoemulsion further comprises:

(1) at least one amphiphilic lipid selected from the group consisting of nonionic amphiphilic lipids, anionic amphiphilic lipids, and mixtures thereof, wherein said anionic amphiphilic lipid or lipids are selected from the group consisting of (a) mixed esters of fatty acids and of fatty alcohols, of carboxylic acids and of glycerol; (b) alkyl ether citrates; (c) alkenyl succinates; (d) fatty esters of phosphoric acid; and (e) mixtures thereof; and

(2) at least one anionic polymer comprising at least one hydrophobic chain, and wherein the weight ratio of the amount of oily phase to the amount of amphiphilic lipid ranges from 1.2 to 10.

In other embodiments, the present invention provides various compositions which contain such a nanoemulsion.

In other embodiments, the present invention provides various methods of using such compositions in topical application to the skin, mucous membranes, scalp, and/or hair.

### DETAILED DESCRIPTION OF THE PREFERRED EMBODIMENTS

Thus, in a first embodiment, the present invention provides oil-in-water nanoemulsions which comprise oily phase globules dispersed in an aqueous phase, wherein said oily phase globules have an average size of less than 100 nm, and wherein said nanoemulsion further comprises:

(1) at least one amphiphilic lipid selected from the group consisting of nonionic amphiphilic lipids, anionic amphiphilic lipids, and mixtures thereof, wherein said anionic amphiphilic lipid or lipids are selected from the group consisting of (a) mixed esters of fatty acids and of fatty alcohols, of carboxylic acids and of glycerol; (b) alkyl ether citrates; (c) alkenyl succinates; (d) fatty esters of phosphoric acid; and (e) mixtures thereof; and

(2) at least one anionic polymer comprising at least one hydrophobic chain, and wherein the weight ratio of the amount of oily phase to the amount of amphiphilic lipid ranges from 1.2 to 10.

The weight ratio of the amount of the oily phase to the amount of the amphiphilic lipid is preferably from 2 to 10, better still from 2 to 6, and even better from 3 to 6.

The nanoemulsions according to the invention generally have a transparent to bluish appearance. Their transparency is measured by a coefficient of transmission at 600 nm ranging from 10 to 90% or alternatively by a turbidity ranging from 60 to 600 NTU, preferably from 70 to 300 NTU, which turbidity is measured with a Hach Model 2100 P portable turbidimeter.

The oil globules of the nanoemulsions of the invention have an average size (number-average size) of less than 100 nm and preferably ranging from 20 to 80 nm and more preferably from 40 to 60 nm. The decrease in the size of the globules makes it possible to promote the penetration of the active principles into the surface layers of the skin (vehicle effect).

The nanoemulsions according to the invention are preferably prepared at temperatures ranging from 4 to 45° C. and are thus compatible with heat-sensitive active principles.

The anionic polymers used according to the present invention are preferably water-soluble or water-dispersible polymers, that is to say that they are soluble in water at a pH of greater than 3.5. They comprise at least one hydrophobic chain, they are non-crosslinked and they preferably have a weight average molecular weight ranging from 10,000 to 2,000,000. These polymers make it possible to increase the viscosity of fluid nanoemulsions (5 cP) by at least a factor of 10. When added to a nanoemulsion, they make it possible to obtain transparent and stable compositions constituting creams. The terms “milk” and “cream” are understood to mean compositions having a viscosity ranging from 0.5 to 150 poises (i.e. 0.05 Pa·s to 15 Pa·s), measured at 25° C. with a Rheomat 180 with a 3, 4, or 5 rotor (according to the viscosity range) at 200 s⁻¹.

Consequently, in another embodiment, the present invention provides a process for thickening an oil-in-water nanoemulsion having oil globules with an average size of less than 100 nm which consists of adding an anionic polymer comprising at least one hydrophobic chain to the nanoemulsion.

The hydrophobic chain or chains of the anionic polymer used according to the present invention are in particular saturated or unsaturated and linear or branched hydrocarbonaceous chains having from 6 to 30 carbon atoms, such as alkyl, arylalkyl, alkylaryl or alkylene; divalent cycloaliphatic groups, such as, in particular, methylenedicyclohexyl and isophorone; or divalent aromatic groups, such as phenylene.

The anionic polymers used in the present invention can be chosen in particular from acrylic or methacrylic acid copolymers, 2-acrylamido-2-methylpropanesulphonic acid copolymers, and mixtures thereof. Acrylic or methacrylic acid copolymers are preferred. The term “copolymers” is understood to mean both copolymers obtained from two kinds of monomers and those obtained from more than two kinds of monomers, such as terpolymers obtained from three kinds of monomers.

The anionic polymers preferably used in the invention are obtained by copolymerization of a monomer (a) chosen from carboxylic acids comprising α,β-monoethylenic unsaturation (monomer a′) and 2-acrylamido-2-methylpropanesulphonic acid (monomer a″) with a nonsurface-active monomer (b) comprising ethylenic unsaturation other than (a) and/or a monomer (c) comprising ethylenic unsaturation resulting from the reaction of an acrylic monomer comprising α,β-monoethylenic unsaturation or of an isocyanate monomer comprising monoethylenic unsaturation with a monohydric nonionic amphiphilic component or with a primary or secondary fatty amine.

Thus, the anionic polymers used in the composition of the invention can be obtained by two synthetic routes:

(1) by copolymerization of the monomers (a′) and (c), or (a′), (b) and (c), or (a″) and (c), or (a″), (b) and (c); or

(2) by modification (and in particular esterification or amidation) of a copolymer formed from the monomers (a′) or from the monomers (a′) and (b), or (a″) and (b), by a monohydric nonionic amphiphilic compound or a primary or secondary fatty amine.

As 2-acrylamido-2-methylpropanesulphonic acid copolymers, mention may in particular be made of those disclosed in the article “Micelle formation of random copolymers of sodium 2-(acrylamido)-2-methylpropanesulfonate and nonionic surfactant macromonomer in water as studied by fluorescence and dynamic light scattering,” Macromolecules, vol. 33, No. 10, pp. 3694-3704 (2000) and in published European patent applications EP-A-0 750 899 and EP-A-1 069 172.

The carboxylic acid comprising α,β-monoethylenic unsaturation constituting the monomer (a′) can be chosen from numerous acids and in particular from acrylic acid, methacrylic acid, crotonic acid, itaconic acid and maleic acid. It is preferably acrylic or methacrylic acid.

The copolymer can comprise a monomer (b) comprising monoethylenic unsaturation which does not have surfactant property. The preferred monomers are those which give water-insoluble polymers when they are homopolymerized. They can be chosen, for example, from C₁-C₄ alkyl acrylates and methacrylates, such as methyl acrylate, ethyl acrylate, butyl acrylate or the corresponding methacrylates. The more particularly preferred monomers are methyl acrylate and ethyl acrylate. The other monomers which can be used are, for example, styrene, vinyltoluene, vinyl acetate, acrylonitrile and vinylidene chloride. Unreactive monomers are preferred, these monomers being those in which the single ethylenic group is the only group which is reactive under the polymerization conditions. However, monomers which comprise groups which react under the effect of heat, such as hydroxyethyl acrylate, can optionally be used.

The monomer (c) is obtained by reaction of an acrylic monomer comprising α,β-monoethylenic unsaturation, such as (a), or of an isocyanate monomer comprising monoethylenic unsaturation with a monohydric nonionic amphiphilic compound or a primary or secondary fatty amine.

The monohydric nonionic amphiphilic compounds or the primary or secondary fatty amines used to produce the nonionic monomer (c) are well known. The monohydric nonionic amphiphilic compounds are generally alkoxylated hydrophobic compounds comprising an alkylene oxide forming the hydrophilic part of the molecule. The hydrophobic compounds are generally composed of an aliphatic alcohol or an alkylphenol, in which a carbonaceous chain comprising at least six carbon atoms constitutes the hydrophobic part of the amphiphilic compound.

The preferred monohydric nonionic amphiphilic compounds are compounds having the following formula (I):

R—(OCH₂CHR′)ₘ—(OCH₂CH₂)ₙ—OH (I)

in which R is chosen from alkyl or alkylene groups comprising from 6 to 30 carbon atoms and alkylaryl groups having alkyl radicals comprising from 8 to 30 carbon atoms, R′ is chosen from alkyl groups comprising from 1 to 4 carbon atoms, n is a mean number ranging from approximately 1 to 150 and m is a mean number ranging from approximately 0 to 50, provided that n is at least as great as m.

Preferably, in the compounds of formula (I), the R group is chosen from alkyl groups comprising from 12 to 26 carbon atoms and alkylphenyl groups in which the alkyl group is C₈-C₁₃; the R′ group is the methyl group; m=0 and n=1 to 25.

The preferred primary and secondary fatty amines are composed of one or two alkyl chains comprising from 6 to 30 carbon atoms.

The monomer used to form the nonionic urethane monomer (c) can be chosen from highly varied compounds. Use may be made of any compound comprising a copolymerizable unsaturation, such as an acrylic, methacrylic or allylic unsaturation. The monomer (c) can be obtained in particular from an isocyanate comprising monoethylenic unsaturation, such as, in particular, α,α-dimethyl-m-isopropenylbenzyl isocyanate.

The monomer (c) can be chosen in particular from acrylates, methacrylates or itaconates of oxyethylenated (1 to 50 EO) C₆-C₃₀ fatty alcohol, such as steareth-20 methacrylate, oxyethylenated (25 EO) behenyl methacrylate, oxyethylenated (20 EO) monocetyl itaconate, oxyethylenated (20 EO) monostearyl itaconate, or the acrylate modified by polyoxyethylenated (25 EO) C₁₂-C₂₄ alcohols; and from dimethyl-m-isopropenylbenzyl isocyanates of oxyethylenated (1 to 50 EO) C₆-C₃₀ fatty alcohol, such as, in particular, the dimethyl-m-isopropenylbenzyl isocyanate of oxyethylenated behenyl alcohol.

According to a specific embodiment of the invention, the anionic polymer is chosen from acrylic terpolymers obtained from (a) a carboxylic acid comprising α,β-ethylenic unsaturation, (b) a nonsurface-active monomer comprising ethylenic unsaturation other than (a), and (c) a nonionic urethane monomer which is the reaction product of a monohydric nonionic amphiphilic compound with an isocyanate comprising monoethylenic unsaturation.

Mention may in particular be made, as anionic polymers comprising at least one hydrophobic chain which can be used in the nanoemulsions of the present invention, of the acrylic acid/ethyl acrylate/alkyl acrylate terpolymer, such as the product which is sold as a 30% aqueous dispersion under the name Acusol 823 by Rohm & Haas; the acrylates/steareth-20 methacrylate copolymer, such as the product which is sold under the name Aculyn 22 by Rohm & Haas; the (meth)acrylic acid/ethyl acrylate/oxyethylenated (25 EO) behenyl methacrylate terpolymer, such as the product which is sold as an aqueous emulsion under the name Aculyn 28 by Rohm & Haas; the acrylic acid/oxyethylenated (20 EO) monocetyl itaconate copolymer, such as the product which is sold as a 30% aqueous dispersion under the name Structure 3001 by National Starch; the acrylic acid/oxyethylenated (20 EO) monostearyl itaconate copolymer, such as the product which is sold as a 30% aqueous dispersion under the name Structure 2001 by National Starch; the acrylates/acrylate modified by polyoxyethylenated (25 EO) C₁₂-C₂₄ alcohols copolymer, such as the 30-32% copolymer latex which is sold under the name Synthalen W2000 by 3V SA; or the methacrylic acid/methyl acrylate/dimethyl-meta-isopropenylbenzyl isocyanate of ethoxylated behenyl alcohol terpolymer, such as the product which as a 24% aqueous dispersion and comprising 40 ethylene oxide groups is disclosed in the published European patent application EP-A-0 173 109.

According to the invention, the anionic polymer or polymers can represent from 0.1 to 10% by weight, preferably from 0.2 to 5% by weight, and more particularly from 1 to 5% by weight, with respect to the total weight of the composition.

The addition of neutralizing agents may prove to be useful in increasing the solubility of the polymers in water. Any known neutralizing agent can then be used, and in particular it can be chosen from inorganic bases, such as sodium hydroxide, potassium hydroxide, or ammonia; and from organic bases, such as mono-, di-, and triethanolamine, aminomethylpropane-1,3-diol, N-methylglucamine, or basic amino acids, such as arginine and lysine, and mixtures thereof. The pH of the compositions according to the invention must be greater than 4 and preferably ranges from 5 to 8 and better still from 5 to 7. The amount of neutralizing agent depends on the polymer used and on the other constituents of the formula. It can range, for example, from 0.01 to 5% by weight, and better still from 0.05 to 5% by weight, of the total weight of the composition.

The anionic polymers used according to the invention can thicken any type of nanoemulsion and in particular the nanoemulsions disclosed in European and French patent applications EP-A-705 593, EP-A-728 460, EP-A-780 114, FR-A-2 787 026, FR-A-2 787 027, FR-A-2 787 325, FR-A-2 787 326, FR-A-2 787 703, FR-A-2 787 728, FR 2 788 007, FR 2 788 449, and FR 2 788 980.

The present nanoemulsions also comprise at least one amphiphilic lipid chosen from nonionic amphiphilic lipids, anionic amphiphilic lipids, as defined above, and their mixtures.

The nonionic amphiphilic lipids of the present invention are preferably chosen from:

1) silicone surfactants;

2) amphiphilic lipids which are liquid at a temperature of less than or equal to 45° C. chosen from esters of at least one polyol and of at least one fatty acid comprising at least one saturated or unsaturated and linear or branched, and in particular unsaturated or branched, C₈-C₂₂ alkyl chain, the polyol being chosen from the group formed by polyethylene glycol comprising from 1 to 60 ethylene oxide units, sorbitan, glycerol possibly comprising from 2 to 30 ethylene oxide units, and polyglycerols comprising from 2 to 15 glycerol units; and

3) esters of fatty acids and of sugars and ethers of fatty alcohols and of sugars;

4) surfactants which are solid at a temperature of less than or equal to 45° C. chosen from glycerol fatty esters, sorbitan fatty esters, and oxyethylenated sorbitan fatty esters, ethoxylated fatty ethers, and ethoxylated fatty esters;

5) block copolymers of ethylene oxide (A) and of propylene oxide (B); and

6) mixtures thereof.

1) The silicone surfactants which can be used according to the present invention are silicone compounds comprising at least one oxyethylene —OCH₂CH₂— chain and/or oxypropylene —OCH₂CH₂CH₂— chain. As silicone surfactants which can be used according to the present invention, mention may be made of those disclosed in U.S. Pat. Nos. 5,364,633 and 5,411,744.

The silicone surfactant used according to the present invention is preferably a compound of formula (II)

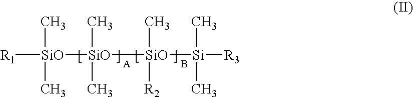

in which:

R₁, R₂, and R₃, each independently of one another, represent a C₁-C₆ alkyl radical or a —(CH₂)ₓ(OCH₂CH₂)_{y}—(OCH₂CH₂CH₂)_{z}—OR₄ radical; at least one R₁, R₂, or R₃ radical not being an alkyl radical; R₄ being a hydrogen, an alkyl radical or an acyl radical;

A is an integer ranging from 0 to 200;

B is an integer ranging from 0 to 50; provided that A and B are not equal to zero at the same time;

x is an integer ranging from 1 to 6;

y is an integer ranging from 1 to 30; and

z is an integer ranging from 0 to 5.

According to a preferred embodiment of the invention, in the compound of formula (I), the alkyl radical is a methyl radical, x is an integer ranging from 2 to 6, and y is an integer ranging from 4 to 30.

As examples of silicone surfactants of formula (II), mention may be made of the compounds of formula (III):

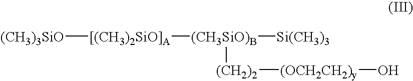

in which A is an integer ranging from 20 to 105, B is an integer ranging from 2 to 10, and y is an integer ranging from 10 to 20.

Mention may also be made, as examples of silicone surfactants of formula (II), of the compounds of formula (IV):

H—(OCH₂CH₂)_{y}—(CH₂)₃—[(CH₃)₂SiO]_{A′}—(CH₂)₃—(OCH₂CH₂)_{y}—OH (IV)

in which A′ and y are integers ranging from 10 to 20.

Use may in particular be made, as silicone surfactants, of those sold by Dow Corning under the names DC 5329, DC 7439-146, DC 2-5695, and Q4-3667. The compounds DC 5329, DC 7439-146, and DC 2-5695 are compounds of formula (III) where respectively A is 22, B is 2, and y is 12; A is 103, B is 10, and y is 12; and A is 27, B is 3, and y is 12.

The compound Q4-3667 is a compound of formula (IV) where A is 15 and y is 13.

2) The amphiphilic lipids which are liquid at a temperature of less than or equal to 45° C. can be chosen in particular from:

polyethylene glycol isostearate with a molecular weight of 400, sold under the name PEG 400 by Unichema;

diglyceryl isostearate, sold by Solvay;

polyglyceryl laurate comprising 2 glycerol units (polyglyceryl-2 laurate), sold under the name diglycerin monolaurate by Solvay;

sorbitan oleate, sold under the name Span 80 by ICI;

sorbitan isostearate, sold under the name Nikkol SI 10R by Nikko;

α-butylglucoside cocoate or α-butylglucoside caprate, sold by Ulice.

3) The esters of fatty acids and of sugars which can be used as nonionic amphiphilic lipids in the nanoemulsions according to the present invention are preferably solid at a temperature of less than or equal to 45° C. and can be chosen in particular from the group consisting of esters or mixtures of esters of a C₈-C₂₂ fatty acid and of sucrose, of maltose, of glucose, or of fructose; and esters or mixtures of esters of a C₁₄-C₂₂ fatty acid and of methylglucose.

The C₈-C₂₂ or C₁₄-C₂₂ fatty acids forming the fatty unit of the esters which can be used in the nanoemulsions of the present invention comprise a saturated or unsaturated linear alkyl chain having from 8 to 22 or from 14 to 22 carbon atoms respectively. The fatty unit of the esters can be chosen in particular from stearates, behenates, arachidonates, palmitates, myristates, laurates, caprates, and their mixtures. Stearates are preferably used.

As examples of esters or of mixtures of esters of a fatty acid and of sucrose, of maltose, of glucose, or of fructose, mention may be made of sucrose monostearate, sucrose distearate, sucrose tristearate and their mixtures, such as the products sold by Croda under the name Crodesta F50, F70, F110, and F160 having respectively an HLB (Hydrophilic Lipophilic Balance) of 5, 7, 11, and 16; and, as examples of esters or of mixtures of esters of a fatty acid and of methylglucose, of the distearate of methylglucose and of polyglycerol-3, sold by Goldschmidt under the name of Tego-care 450. Mention may also be made of glucose or maltose monoesters, such as methyl O-hexadecanoyl-6-D-glucoside and methyl O-hexadecanoyl-6-D-maltoside.

The ethers of fatty alcohols and of sugars which can be used as nonionic amphiphilic lipids in the nanoemulsions according to the present invention are solid at a temperature of less than or equal to 45° C. and can be chosen in particular from the group consisting of ethers or mixtures of ethers of C₈-C₂₂ fatty alcohols and of glucose, of maltose, of sucrose, or of fructose; and ethers or mixtures of ethers of C₁₄-C₂₂ fatty alcohols and of methylglucose. They are in particular alkylpolyglucosides.

The C₈-C₂₂ or C₁₄-C₂₂ fatty alcohols forming the fatty unit of the ethers which can be used in the nanoemulsions of the present invention comprise a saturated or unsaturated linear alkyl chain having from 8 to 22 or from 14 to 22 carbon atoms, respectively. The fatty unit of the ethers can be chosen in particular from the decyl, cetyl, behenyl, arachidyl, stearyl, palmityl, myristyl, lauryl, capryl or hexadecyl units and their mixtures, such as cetearyl.

As examples of ethers of fatty alcohols and of sugars, mention may be made of alkylpolyglucosides, such as decylglucoside and laurylglucoside, sold, for example, by Henkel under the respective names of Plantaren 2000 and Plantaren 1200, cetostearylglucoside, optionally as a mixture with cetostearyl alcohol, sold, for example, under the name Montanov 68 by Seppic, under the name Tego-care CG90 by Goldschmidt and under the name Emulgade KE3302 by Henkel, and arachidylglucoside, for example in the form of a mixture of arachidyl and behenyl alcohols and of arachidylglucoside sold under the name Montanov 202 by Seppic.

As nonionic amphiphilic lipids of this type, use is more particularly made of sucrose monostearate, sucrose distearate, sucrose tristearate and their mixtures, the distearate of methylglucose and of polyglycerol-3, and alkylpolyglucosides.

4) The glycerol fatty esters which can be used as nonionic amphiphilic lipids in the nanoemulsions of the present invention, which are solid at a temperature of less than or equal to 45° C., can be chosen in particular from the group consisting of the esters formed of at least one acid comprising a saturated linear alkyl chain having from 16 to 22 carbon atoms and of 1 to 10 glycerol units. Use may be made of one or more of these glycerol fatty esters in the nanoemulsion of the invention.

These esters can be chosen in particular from stearates, behenates, arachidates, palmitates or their mixtures. Stearates and palmitates are preferably used.

As examples of surfactants which can be used in the nanoemulsions of the present invention, mention may be made of decaglycerol monostearate, distearate, tristearate, and pentastearate (10 glycerol units) (CTFA names: Polyglyceryl-10 stearate, Polyglyceryl-10 distearate, Polyglyceryl-10 tristearate, and Polyglyceryl-10 pentastearate), such as the products sold under the respective names Nikkol Decaglyn 1-S, 2-S, 3-S, and 5-S by Nikko; and diglyceryl monostearate (CTFA name: Polyglyceryl-2 stearate), such as the product sold by Nikko under the name Nikkol DGMS.

The sorbitan fatty esters which can be used as nonionic amphiphilic lipids in the nanoemulsions according to the present invention, which are solid at a temperature of less than or equal to 45° C., are chosen in particular from the group consisting of esters of C₁₆-C₂₂ fatty acid and of sorbitan and oxyethylenated esters of C₁₆-C₂₂ fatty acids and of sorbitan. They are formed of at least one fatty acid comprising at least one saturated linear alkyl chain, having respectively from 16 to 22 carbon atoms, and of sorbitol or of ethoxylated sorbitol. The oxyethylenated esters generally comprise from 1 to 100 ethylene oxide units and preferably from 2 to 40 ethylene oxide (EO) units.

These esters can be chosen in particular from stearates, behenates, arachidates, palmitates, and their mixtures. Stearates and palmitates are preferably used.

As examples of sorbitan fatty esters and of oxyethylenated sorbitan fatty esters which can be used in the nanoemulsions of the present invention, mention may be made of sorbitan monostearate (CTFA name: sorbitan stearate), sold by ICI under the name Span 60; sorbitan monopalmitate (CTFA name: sorbitan palmitate), sold by ICI under the name Span 40; or sorbitan 20 EO tristearate (CTFA name: Polysorbate 65), sold by ICI under the name Tween 65.

The ethoxylated fatty ethers which are solid at a temperature of less than or equal to 45° C. which can be used as nonionic amphiphilic lipids in the nanoemulsions according to the present invention are preferably ethers formed of 1 to 100 ethylene oxide units and of at least one fatty alcohol chain having from 16 to 22 carbon atoms. The fatty chain of the ethers can be chosen in particular from the behenyl, arachidyl, stearyl, or cetyl units and their mixtures, such as cetearyl. As examples of ethoxylated fatty ethers, mention may be made of the ethers of behenyl alcohol comprising 5, 10, 20, and 30 ethylene oxide units (CTFA names: Beheneth-5, Beheneth-10, Beheneth-20, and Beheneth-30), such as the products sold under the names Nikkol BB5, BB10, BB20, and BB30 by Nikko; and the ether of stearyl alcohol comprising 2 ethylene oxide units (CTFA name: Steareth-2), such as the product sold under the name Brij 72 by ICI.

The ethoxylated fatty esters which are solid at a temperature of less than or equal to 45° C. which can be used as nonionic amphiphilic lipids in the nanoemulsions according to the present invention are esters formed of 1 to 100 ethylene oxide units and of at least one fatty acid chain comprising from 16 to 22 carbon atoms. The fatty chain of the esters can be chosen in particular from the stearate, behenate, arachidate, or palmitate units and their mixtures. As examples of ethoxylated fatty esters, mention may be made of the ester of stearic acid comprising 40 ethylene oxide units, such as the product sold under the name Myrj 52 (CTFA name: PEG-40 stearate) by ICI; and the ester of behenic acid comprising 8 ethylene oxide units (CTFA name: PEG-8 behenate), such as the product sold under the name Compritol HD5 ATO by Gattefosse.

5) The block copolymers of ethylene oxide (A) and of propylene oxide (B) which can be used as nonionic amphiphilic lipids in the nanoemulsions according to the present invention can be chosen in particular from the block copolymers of formula (V):

HO(C₂H₄O)ₓ(C₃H₆O)_{y}(C₂H₄O)_{z}H (V)

in which x, y, and z are integers such that x+z ranges from 2 to 100 and y ranges from 14 to 60, and their mixtures and more particularly from the block copolymers of formula (V) having an HLB ranging from 2 to 16.

These block copolymers can be chosen in particular from poloxamers and in particular from Poloxamer 231, such as the product sold by ICI under the name Pluronic L81 of formula (V) with x=z=6, y=39 (HLB 2); Poloxamer 282, such as the product sold by ICI under the name Pluronic L92 of formula (V) with x=z=10, y=47 (HLB 6); and Poloxamer 124, such as the product sold by ICI under the name Pluronic L44 of formula (V) with x=z=11, y=21 (HLB 16).

As nonionic amphiphilic lipids, mention may also be made of the mixtures of nonionic surfactants disclosed in published European patent application EP-A-705 593, which is incorporated herein by reference.

Among nonionic amphiphilic lipids, use may in particular be made of:

PEG 400 isostearate or PEG-8 isostearate (comprising 8 mol of ethylene oxide),

diglyceryl isostearate,

polyglyceryl monolaurate comprising 2 glycerol units and polyglyceryl stearates comprising 10 glycerol units,

sorbitan oleates,

sorbitan isostearate,

and mixtures thereof.

The anionic amphiphilic lipids which can be used in the nanoemulsions of the present invention can be chosen from:

1) mixed esters of fatty acids or of fatty alcohols, of carboxylic acids and of glycerol;

2) alkyl ether citrates;

3) alkenyl succinates chosen from alkoxylated alkenyl succinates, alkoxylated glucose alkenyl succinates, and alkoxylated methylglucose alkenyl succinates; and

4) phosphoric acid fatty esters.

1) The mixed esters of fatty acids or of fatty alcohols, of carboxylic acids and of glycerol which can be used as anionic amphiphilic lipids in the nanoemulsions according to the present invention can be chosen in particular from the group consisting of mixed esters of fatty acids or of fatty alcohols having an alkyl chain comprising from 8 to 22 carbon atoms and of α-hydroxy acids and/or of succinic acid with glycerol. The α-hydroxy acid can be, for example, citric acid, lactic acid, glycolic acid, malic acid and their mixtures.

The alkyl chain of the fatty acids or alcohols from which the mixed esters which can be used in the nanoemulsions of the present invention derive can be saturated or unsaturated and linear or branched. It can in particular b e a stearate, isostearate, linoleate, oleate, behenate, arachidonate, palmitate, myristate, laurate, caprate, isostearyl, stearyl, linoleyl, oleyl, behenyl, myristyl, lauryl, or capryl group or a mixture thereof.

As examples of mixed esters which can be used in the nanoemulsions of the present invention, mention may be made of the mixed ester of glycerol and of the mixture of citric, lactic, linoleic and oleic acids (CTFA name: Glyceryl citrate/lactate/linoleate/oleate) sold by Hüls under the name Imwitor 375; the mixed ester of succinic acid and of isostearyl alcohol with glycerol (CTFA name: Isostearyl diglyceryl succinate) sold by Hüls under the name Imwitor 780 K; the mixed ester of citric acid and of stearic acid with glycerol (CTFA name: Glyceryl stearate citrate) sold by Hüls under the name Imwitor 370; or the mixed ester of lactic acid and of stearic acid with glycerol (CTFA name: Glyceryl stearate lactate) sold by Danisco under the name Lactodan B30 or Rylo LA30.

2) The alkyl ether citrates which can be used as anionic amphophilic lipids in the nanoemulsions according to the present invention can be chosen in particular from the group consisting of the monoesters, diesters, or triesters formed by citric acid and at least one oxyethylenated fatty alcohol, comprising a saturated or unsaturated and linear or branched alkyl chain having from 8 to 22 carbon atoms and comprising from 3 to 9 ethylene oxide groups, and their mixtures. This is because it is possible to use a mixture of one or more of these citrates in the nanoemulsions of the present invention.

These citrates can be chosen, for example, from the mono-, di-, and triesters of citric acid and of ethoxylated lauryl alcohol, comprising from 3 to 9 ethylene oxide groups, sold by Witco under the name Witconol EC, in particular Witconol EC 2129, which is predominantly a dilaureth-9 citrate, and Witconol EC 3129, which is predominantly a trilaureth-9 citrate.

The alkyl ether citrates used as anionic amphophilic lipids are preferably employed in the form neutralized to a pH of approximately 7, the neutralizing agent being chosen from inorganic bases, such as sodium hydroxide, potassium hydroxide, or ammonia; and organic bases, such as mono-, di-, and triethanolamine, aminomethylpropane-1,3-diol, N-methylglucamine, or basic amino acids, such as arginine and lysine, and mixtures thereof.

3) The alkenyl succinates which can be used as anionic amphiphilic lipids in the nanoemulsions of the present invention are in particular ethoxylated and/or propoxylated derivatives and they are preferably chosen from the compounds of formulae (VI) or (VII):

HOOC—(HR)C—CH₂—COO—E (VI)

HOOC—(HR)C—CH₂—COO—E—O—CO—CH₂—C(HR′)—COOH (VII)

in which:

the R and R′ radicals are chosen from linear or branched alkylene radicals comprising from 6 to 22 carbon atoms; and

E is chosen from oxyethylene chains of formula (C₂H₄O)ₙ in which n ranges from 2 to 100; oxypropylene chains of formula (C₃H₆O)_{n′}, in which n′ ranges from 2 to 100; random or block copolymers comprising oxyethylene chains of formula (C₂H₄O)ₙ and oxypropylene chains of formula (C₃H₆O)_{n′}, such that the sum of n and n′ ranges from 2 to 100; oxyethylenated and/or oxypropylenated glucose groups comprising, on average, from 4 to 100 oxyethylene and/or oxypropylene units distributed over all the hydroxyl functional groups; and oxyethylenated and/or oxypropylenated methylglucose groups comprising, on average, from 4 to 100 oxyethylene and/or oxypropylene units distributed over all the hydroxyl functional groups.

In the formulae (VI) and (VII), n and n′ are mean values and are therefore not necessarily integers. The choice is advantageously made, for n, of a value ranging from 5 to 60 and more preferably still from 10 to 30.

The R and/or R′ radical is advantageously chosen from linear alkylene radicals comprising from 8 to 22 and preferably from 14 to 22 carbon atoms. It can, for example, be the hexadecenyl radical, comprising 16 carbon atoms, or the octadecenyl radical, comprising 18 carbon atoms.

The compounds of formulae (VI) and (VII) described above in which E is chosen from oxyethylene chains, oxypropylene chains, and copolymers comprising oxyethylene chains and oxypropylene chains can be prepared in accordance with the description which is given in published patent applications WO-A-94/00508, EP-A-1 071 99, and GB-A-2 131 820, which are incorporated herein by reference.

The acid functional group —COOH of the anionic amphiphilic lipids of formulae (VI) and (VII) is generally found in the nanoemulsions of the present invention in the form neutralized by a neutralizing agent, the neutralizing agent being chosen, for example, from inorganic bases, such as sodium hydroxide, potassium hydroxide, or ammonia; or organic bases, such as mono-, di-, and triethanolamine, aminomethylpropane-1,3-diol, N-methylglucamine, or basic amino acids, such as arginine and lysine, and their mixtures.

As examples of anionic amphiphilic lipid of this type which can be used in the nanoemulsions of the present invention, mention may be made of hexadecenyl succinate 18 EO (compound of formula VI with R=hexadecenyl, E=(C₂H₄O)ₙ and n=18); hexadecenyl succinate 45 EO (compound of formula VI with R=hexadecenyl, E=(C₂H₄O)ₙ and n=45); dihexadecenyl succinate 18 EO (compound of formula VII with R=R′=hexadecenyl, E=(C₂H₄O)ₙ and n=18); dihexadecenyl succinate of glucose 10 EO (compound of formula VII with R=R′=hexadecenyl and E=oxyethylenated glucose comprising 10 oxyethylene groups); dihexadecenyl succinate of glucose 20 EO (compound of formula VII with R=R′=hexadecenyl and E=oxyethylenated glucose comprising 20 oxyethylene groups); dioctadecenyl succinate of methylglucose 20 EO (compound of formula VII with R=R′=octadecenyl and E=oxyethylenated methylglucose comprising 20 oxyethylene groups); and mixtures thereof.

4) The phosphoric acid fatty esters and their oxyethylenated derivatives which can be used as anionic amphophilic lipids in the nanoemulsions according to the present invention can be chosen in particular from the group consisting of the esters formed of phosphoric acid and of at least one alcohol comprising a saturated or unsaturated and linear or branched alkyl chain having from 8 to 22 carbon atoms and the esters formed of phosphoric acid and of at least one ethoxylated alcohol comprising a saturated or unsaturated and linear or branched alkyl chain having from 8 to 22 carbon atoms and comprising from 2 to 40 oxyethylene groups, their salts and their mixtures. This is because it is possible to use a mixture of one or more of these phosphoric acid esters in the nanoemulsions of the present invention.

These esters can be chosen in particular from esters of phosphoric acid and of C₉-C₁₅ alcohols or their salts, such as the potassium salt of C₉-C₁₅ alkyl phosphate sold under the name Arlatone MAP by ICI; esters of phosphoric acid and of stearyl and/or isostearyl alcohols, such as the phosphate of stearyl/isostearyl alcohols (CTFA name: Octyldecyl phosphate) sold under the name Hostaphat CG120 by Hoechst Celanese; esters of phosphoric acid and of cetyl alcohol, and their oxyethylenated derivatives, such as the product sold under the name Crodafos CES (mixture of cetearyl alcohol, of dicetyl phosphate, and of ceteth-10 phosphate) by Croda; or esters of phosphoric acid and of tridecyl alcohol, and their oxyethylenated derivatives, such as the product sold under the name Crodafos T10 (CTFA name: Trideceth-10 phosphate) by Croda. The oxyethylenated derivatives of phosphoric acid and of fatty alcohol can be prepared in accordance with the description given in published International patent application WO-A-96/14145, the content of which is incorporated herein by reference.

These phosphoric acid fatty esters are preferably employed in the form neutralized to a pH of approximately 7, the neutralizing agent being chosen from inorganic bases, such as sodium hydroxide, potassium hydroxide, or ammonia; and organic bases, such as mono-, di-, and triethanolamine, aminomethylpropane-1,3-diol, N-methylglucamine, or basic amino acids such as arginine and lysine, and mixtures theeof.

Depending on whether it is more hydrophilic or more lipophilic in nature, the nonionic or anionic amphiphilic lipid can be introduced into the aqueous phase or into the oily phase of the nanoemulsion. The amount of amphiphilic lipid ranges from 0.2 to 15% by weight, and preferably from 1 to 10% by weight, with respect to the total weight of the nanoemulsion.

The weight ratio of the amount of the oily phase to the amount of amphiphilic lipid ranges from 1.2 to 10 and preferably from 1.2 to 6. The term “amount of oily phase” is understood here to mean the total amount of the constituents of this phase, excluding the amount of amphiphilic lipid.

According to a specific embodiment of the invention, the nanoemulsions of the present invention can moreover comprise one or more additional anionic amphiphilic lipids. Their addition, as additives, may further improve the stability of the dispersion.

Thus, the additional anionic amphiphilic lipids which can be used in the nanoemulsions of the present invention are preferably chosen from:

alkaline salts of dicetyl and dimyristyl phosphate;

alkaline salts of cholesterol sulphate;

alkaline salts of cholesterol phosphate;

lipoamino acids and their salts, such as mono- and disodium acylglutamates, for instance the disodium salt of N-stearoyl-L-glutamic acid sold under the name Acylglutamate HS21 by Ajinomoto;

sodium salts of phosphatidic acid;

phospholipids;

alkylsulphonic derivatives, in particular of formula (VIII):

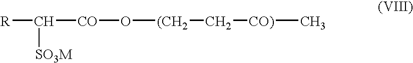

in which:

R represents a C₁₆-C₂₂ alkyl radical, in particular the C₁₆H₃₃ and C₁₈H₃₇ radicals taken as a mixture or separately; and

M is an alkali metal or an alkaline earth metal, such as sodium; and mixtures thereof.

When the nanoemulsion comprises one or more additional anionic amphiphilic lipids, they are present in the nanoemulsions of the present invention preferably in concentrations ranging from 0.01 to 10% by weight, and more particularly from 0.2 to 1% by weight, with respect to the total weight of the nanoemulsion.

The oily phase of the nanoemulsions according to the present invention comprise at least one oil. The oils which can be used in the nanoemulsions of the present invention are preferably chosen from the group consisting of:

oils of animal or vegetable origin, formed by esters of fatty acids and of polyols, in particular liquid triglycerides, for example sunflower, maize, soybean, avocado, jojoba, gourd, grape seed, sesame and hazelnut oils, fish oils or glycerol tricaprate/caprylate, or vegetable or animal oils of formula R₉COOR₁₀ in which R₉ represents the residue of a higher fatty acid comprising from 7 to 29 carbon atoms and R₁₀ represents a linear or branched hydrocarbonaceous chain comprising from 3 to 30 carbon atoms, in particular an alkyl or alkenyl chain, for example Purcellin oil or liquid jojoba wax;

natural or synthetic essential oils, such as, for example, eucalyptus, lavandin, lavender, vetiver, litsea cubeba, lemon, sandalwood, rosemary, camomile, savoury, nutmeg, cinnamon, hyssop, caraway, orange, geraniol, cade and bergamot oils;

synthetic oils, such as parleam oil, polyolefins and liquid carboxylic acid esters;

mineral oils, such as hexadecane, isohexadecane and liquid paraffin;

halogenated oils, in particular fluorocarbons, such as fluoroamines, for example perfluorotributylamine, fluorinated hydrocarbons, for example perfluorodecahydronaphthalene, fluoroesters and fluoroethers; and

volatile and nonvolatile silicone oils.

The polyolefins which can be used as synthetic oils are in particular poly-α-olefins and more particularly those of hydrogenated or nonhydrogenated polybutene type and preferably hydrogenated or nonhydrogenated polyisobutene type.

The liquid carboxylic acid esters which can be used as synthetic oils can be esters of mono-, di-, tri-, or tetracarboxylic acids. The total carbon number of the esters is generally greater than or equal to 10 and preferably less than 100 and more particularly less than 80. They are in particular monoesters of saturated or unsaturated and linear or branched C₁-C₂₆ aliphatic acids and of saturated or unsaturated and linear or branched C₁-C₂₆ aliphatic alcohols, the total carbon number of the esters generally being greater than or equal to 10. Use may also be made of esters of C₄-C₂₂ di- or tricarboxylic acids and of C₁-C₂₂ alcohols, and esters of mono-, di-, or tricarboxylic acids and of C₂-C₂₆ di-, tri-, tetra-, or pentahydroxyl alcohols.

It is preferable, among the above-mentioned esters, to use alkyl palmitates, such as ethyl palmitate, isopropyl palmitate, 2-ethylhexyl palmitate, or 2-octyldecyl palmitate; alkyl myristates, such as isopropyl myristate, butyl myristate, cetyl myristate, or 2-octyldodecyl myristate; alkyl stearates, such as hexyl stearate, butyl stearate, or isobutyl stearate; alkyl malates such as dioctyl malate; alkyl laurates, such as hexyl laurate and 2-hexyldecyl laurate; isononyl isononanoate; or cetyl octanoate.

The nanoemulsions in accordance with the present invention comprise an amount of oily phase (oil and other fatty substances apart from the amphiphilic lipid) preferably ranging from 2 to 40% by weight, more particularly from 4 to 30% by weight, and preferably from 4 to 20% by weight, with respect to the total weight of the nanoemulsion.

The nanoemulsions in accordance with the present invention can further comprise one or more solvents, in particular for improving, if necessary, the transparency of the composition.

These solvents are preferably chosen from the group consisting of:

lower C₁-C₈ alcohols, such as ethanol;

glycols, such as glycerol, propylene glycol, 1,3-butylene glycol, dipropylene glycol, or polyethylene glycols comprising from 4 to 16 ethylene oxide units and preferably from 8 to 12; and

sugars, such as glucose, fructose, maltose, lactose, or sucrose.

These additives can be used as a mixture. When they are present in the nanoemulsions of the present invention, they can be used at concentrations preferably ranging from 0.01 to 30% by weight, and better still from 5 to 20% by weight, with respect to the total weight of the nanoemulsion. The amount of alcohol(s) and/or of sugar(s) preferably ranges from 5 to 20% by weight with respect to the total weight of the nanoemulsion, and the amount of glycol(s) preferably ranges from 5 to 15% by weight with respect to the total weight of the nanoemulsion.

The process for the preparation of a nanoemulsion according to the present invention involves mixing the aqueous phase and the oily phase, with vigorous stirring, at an ambient temperature of less than 45° C., carrying out a stage of high-pressure homogenization at a pressure of greater than 5×10⁷ Pa, and then adding the polymer used according to the invention. According to a preferred embodiment of the invention, a further stage of high-pressure homogenization is subsequently carried out at a pressure of greater than 5×10⁷ Pa. The high-pressure homogenization is preferably carried out at a pressure ranging from 6×10⁷ to 18×10⁷ Pa. The shearing preferably ranges from 2×10⁶ s⁻¹ to 5×10⁸ s⁻¹ and better still from 1×10⁸ s⁻¹ to 3×10⁸ s⁻¹ (s⁻¹ means second⁻¹). Such a process makes it possible to prepare, at ambient temperature, nanoemulsions which are compatible with heat-sensitive active compounds and which can comprise oils and in particular fragrances which include fatty substances, without denaturing them.

The nanoemulsions according to the present invention can be used in any field where this type of composition is of use. They can constitute in particular compositions for topical use and in particular cosmetic or dermatological compositions, according to the type of active principles and the amount of these active principles comprised therein. They can also be used as ophthalmic vehicles. In addition, they can constitute, in the pharmaceutical field, the vehicle for a pharmaceutical composition which can be administered orally, parenterally, or transcutaneously.

Such a composition for topical, pharmaceutical, or ophthalmic use comprises a physiologically acceptable medium, that is to say a medium compatible with the skin, mucous membranes, scalp, eyes, and/or hair.

Thus, another embodiment of the present invention is an ophthalmic vehicle, characterized in that it comprises a nanoemulsion as defined above.

Another embodiment of the present invention is a pharmaceutical composition, characterized in that it comprises a nanoemulsion as defined above.

Another embodiment of the present invention is a cosmetic or dermatological composition, characterized in that it comprises a nanoemulsion as defined above.

The compositions of the invention can comprise adjuvants and in particular water-soluble or fat-soluble active principles having a cosmetic or dermatological activity. The fat-soluble active principles are present in the oily globules of the emulsion, whereas the water-soluble active principles are present in the aqueous phase of the emulsion. As examples of active principles, mention may be made of vitamins and their derivatives, such as vitamin E and its esters, such as vitamin E acetate, vitamin C and its esters, vitamins B, vitamin A alcohol or retinol and its esters, such as vitamin A palmitate, or vitamin A acid or retinoic acid and its derivatives; provitamins, such as panthenol, niacinamide or ergocalciferol; antioxidants; essential oils; humectants; sunscreen agents; moisturizing agents; proteins; ceramides; and pseudoceramides. Mention may be also be made, as adjuvants, of sequestering agents, softeners, colouring materials (pigments or dyes) and fragrances.

As ophthalmic active principles, mention may be made of, for example, antiglaucoma agents, such as betaxolol; antibiotics, such as acyclovir; antiallergics; anti-inflammatory agents, such as ibuprofen and its salts, diclofenac and its salts, or indomethacin; or antiviral agents.

The amounts of these various adjuvants are those conventionally used in the field under consideration, for example from 0.01 to 20% by weight of the total weight of the composition. These adjuvants and their concentrations must be such that they do not modify the property desired for the composition of the invention.

The nanoemulsions of the present invention can be used, for example, for caring for, treating or making up the skin of the face, the boby and/or the scalp.

Another embodiment of the present invention is therefore the cosmetic use of the nanoemulsion as defined above for caring for, treating and/or making up the skin.

In addition, the nanoemulsions of the present invention can also be used for caring for and/or treating the hair, i.e., they make it possible to obtain a deposit of oil do the hair which gives it greater sheen and renders it more resistant to styling, without, however, rendering it lank. They also make it possible, in pretreatment, to improve the effects of dyeing or of permanent waving.

Thus, another embodiment of the present invention is the use of the nanoemulsion as defined above for caring for and/or treating the hair.

The nanoemulsions according to the present invention make possible, in particular, good moisturizing of the skin, mucous membranes and/or scalp and is particularly suitable for the treatment of dry skin.

Another embodiment of the present invention is, thus, a process for caring for and/or moisturizing the skin, mucous membranes and/or scalp, characterized in that a nanoemulsion as defined above is applied to the skin, mucous membranes and/or scalp.

As noted above, the present compositions may be used for treating, protecting, caring for, removing make-up from and/or cleansing the skin, the lips, the scalp, and/or the hair, and/or of making up the skin and/or the lips by applying the composition to the skin, lips, scalp, and/or hair. When using the present composition in such a manner, the composition is suitably applied to the skin, lips, scalp, and/or hair in an amount of 0.1 to 5 mg/cm², preferably 0.5 to 4 mg/cm², more preferably 1 to 3 mg/cm² (these numbers being approximate). The amount is generally 2 mg/cm².

The present invention also relates to the use of a nanoemulsion according to the invention in the manufacture of composition intended for the treatment of dry skin.

Finally, the present invention also relates to the use of a nanoemulsion according to the invention in the manufacture of an ophthalmological composition.

Other features of the invention will become apparent in the course of the following descriptions of exemplary embodiments which are given for illustration of the invention and are not intended to be limiting thereof.

### EXAMPLES

In the following examples, and throughout this specification, all amounts are % by weight, and all temperatures are in degrees Celsius, unless expressly stated to be otherwise.

### Example 1

Cream

| Phase A: | |
|---|---|
| PEG 400 isostearate | 4.5% |
| Disodium acylglutamate | 0.5% |
| Isopropyl myristate | 5 |
| Isocetyl stearate | 10% |
| Phase B: | |
| Dipropylene glycol | 10% |
| Glycerol | 5% |
| Distilled water | 45% |
| Phase C: | |
| Methacrylic acid/methyl | 0.87% |
| acrylate/dimethyl-m-isopropenylbenzyl | |
| isocyanate of ethoxylated behenyl | |
| alcohol comprising 40 EO terpolymer, as | |
| a 24% aqueous dispersion | |
| (i.e. 0.2% of active material) | |
| Triethanolamine | 0.14% |
| Distilled water | 18.99% |

Procedure: The nanoemulsion is prepared in the high-pressure homogenizer from phases A and B. Phase C is introduced into the nanoemulsion while stirring with the deflocculator. The combined mixture is passed to the high-pressure homogenizer under the same conditions.

A cream is obtained which has a turbidity of 170 NTU, a viscosity of 3.84 Pa·s (rotor 3, at 200 s⁻¹) and a pH of approximately 7. This cream spreads easily over the skin and is pleasant to use.

### Example 2

Cream

| Phase A: | |
|---|---|
| PEG 400 isostearate | 4.5% |
| Disodium acylglutamate | 0.5% |
| Isopropyl myristate | 5% |
| Isocetyl stearate | 10% |
| Phase B: | |
| Dipropylene glycol | 10% |
| Glycerol | 5% |
| Distilled water | 37.5% |
| Phase C: | |
| 30-32% latex of acrylate/acrylate | 0.968% |
| modified by polyoxyethylenated (25 EO) | |
| C₁₂-C₂₄ alcohols copolymer (Synthalen | |
| W2000 from 3V SA) | |
| Triethanolamine | 0.194% |
| Distilled water | 26.338% |

Procedure: The nanoemulsion is prepared in the high-pressure homogenizer from phases A and B. Phase C is introduced into the nanoemulsion while stirring with the deflocculator. The combined mixture is passed to the high-pressure homogenizer under the same conditions.

A cream is obtained which has a turbidity of 284 NTU, a viscosity of 0.5 Pa·s (rotor 3, at 200 s⁻¹), and a pH of approximately 7. This cream spreads easily over the skin and is pleasant to use.

### Comparative Example

| Phase A: | |
|---|---|
| PEG 400 isostearate | 4.5% |
| Disodium acylglutamate | 0.5% |
| Isopropyl myristate | 5% |
| Isocetyl stearate | 10% |
| Phase B: | |
| Dipropylene glycol | 10% |
| Glycerol | 5% |
| Distilled water | 45% |
| Phase C: | |
| Carboxyvinyl polymer (Carbopol 980) | 0.26% |
| Triethanolamine | 0.39% |
| Distilled water | 19.35% |

The emulsion is prepared with the high-pressure homogenizer from phases A and B. Phase C is introduced into the emulsion while stirring with the deflocculator. The composition obtained has a pH of approximately 7. This is white cream and not a transparent composition.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An oil-in-water nanoemulsion, comprising oily phase globules dispersed in an aqueous phase, wherein said oily phase globules have an average size of less than 100 nm, and wherein said nanoemulsion further comprises:
(1) at least one amphiphilic lipid selected from the group consisting of nonionic amphiphilic lipids, anionic amphiphilic lipids, and mixtures thereof, wherein said anionic amphiphilic lipid or lipids are selected from the group consisting of (a) mixed esters of fatty acids and of fatty alcohols, of carboxylic acids and of glycerol; (b) alkyl ether citrates; (c) alkenyl succinates; (d) fatty esters of phosphoric acid; and (e) mixtures thereof; and
(2) at least one anionic polymer comprising at least one hydrophobic chain, and wherein the weight ratio of the amount of oily phase to the amount of amphiphilic lipid ranges from 1.2 to 10.

2. A nanoemulsion according to claim 1, wherein said weight ratio of the amount of oily phase to the amount of amphiphilic lipid ranges from 2 to 6.

3. A nanoemulsion according to claim 1, wherein said oily phase globules have a mean size ranging from 20 to 80 nm.

4. A nanoemulsion according to claim 1, wherein said anionic polymer is water-soluble or water-dispersible.

5. A nanoemulsion according to claim 1, wherein said hydrophobic chain of said anionic polymer is selected from the group consisting of saturated or unsaturated and linear or branched hydrocarbonaceous chains having from 6 to 30 carbon atoms, divalent cycloaliphatic groups, and divalent aromatic groups.

6. A nanoemulsion to claim 1, wherein said hydrophobic chain of said anionic polymer is selected from the group consisting of alkyl, arylalkyl, alkylaryl, alkylene, methylenedicyclohexyl, isophorone, and phenylene chains.

7. A nanoemulsion according to claim 1, wherein said anionic polymer has a molecular weight ranging from 10,000 to 2,000,000.

8. A nanoemulsion according to claim 1, wherein said anionic polymer is selected from the group consisting of acrylic or methacrylic acid copolymers, 2-acrylamido-2-methylpropanesulphonic acid copolymers, and mixtures thereof.

9. A nanoemulsion according to claim 1, wherein said anionic polymer is obtained by copolymerization of a monomer (a) chosen from carboxylic acids comprising α,β-ethylenic unsaturation (monomer a′) and 2-acrylamido-2-methylpropanesulphonic acid (monomer a″) with a non-surface-active monomer (b) comprising ethylenic unsaturation other than (a) and/or a monomer (c) comprising ethylenic unsaturation resulting from the reaction of an acrylic monomer comprising α,β-monoethylenic unsaturation or of an isocyanate monomer comprising monaethylenic unsaturation with a monohydric nonionic amphiphilic component or with a primary or secondary fatty amine.

10. A nanoemulsion according to claim 1, wherein said anionic polymer is an acrylic terpolymer obtained from polymerization of (a) a carboxylic acid comprising α,β-ethylenic unsaturation, (b) a non-surface-active monomer comprising ethylenic unsaturation other than (a), and (c) a nonionic urethane monomer which is the reaction product of a monohydric nonionic amphiphilic compound with an isocyanate comprising monoethylenic unsaturation.

11. A nanoemulsion according to claim 1, wherein said anionic polymer is selected from the group consisting of acrylic acid/ethyl acrylate/alkyl acrylate terpolymers, acrylates/steareth-20 methacrylate copolymers, (meth)acrylic acid/ethyl acrylate/oxyethylenated (25 EO) behenyl methacrylate terpolymers, acrylic acid/oxyethylenated (20 EO) monocetyl itaconate copolymers, acrylic acid/oxyethylenated (20 EO) monostearyl itaconate copolymers, acrylates/acrylate modified by polyoxyethylenated (25 EO) C₁₂-C₂₄ alcohols copolymers, methacrylic acid/methyl acrylate/dimethyl-meta-isopropenylbenzyl isocyanate of ethoxylated behenyl alcohol terpolymers, and mixtures thereof.

12. A nanoemulsion according to claim 1, wherein the amount of said anionic polymer(s) ranges from 0.1 to 10% by weight with respect to the total weight of the composition.

13. A nanoemulsion according to claim 1, wherein said nonionic amphiphilic lipid is selected from the group consisting of
(1) silicone surfactants;
(2) amphiphilic lipids which are liquid at a temperature of less than or equal to 45° C. selected from the group consisting of esters of at least one polyol and of at least one fatty acid comprising at feast one saturated or unsaturated and linear or branched C₈-C₂₂ alkyl chain;
(3) esters of fatty acid and of sugar and ethers of fatty alcohol and of sugar;
(4) surfactants which are solid at a temperature of less than or equal to 45° C. selected from the group consisting of glycerol fatty esters, sorbitat fatty esters and oxyethylenated sorbitan fatty esters, ethoxylated fatty ethers and ethoxylated fatty esters, 5) block copolymers of ethylene oxide (A) and of propylene oxide (B); and
mixtures thereof.

14. A nanoemulsion according to claim 1, wherein said amount of amphiphilic lipid ranges from 0.2 to 15% by weight with respect to the total weight of the composition.

15. A nanoemulsion according to claim 1, further comproising at least one additional anionic amphiphilic lipid selected from the group consisting of alkaline salts of dicetyl and dimyristyl phosphate; alkaline salts of cholesterol sulphate; alkaline salts of cholesterol phosphate; lipoamino acids and their salts; sodium salts of phosphatidic acid; phospholipids; and alkylsulphonic derivatives of formula: 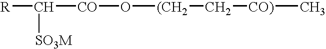
in which R represents a C₁₆-C₂₂ alkyl radical and M is an alkali metal or an alkaline earth metal.

16. A nanoemulsion according to claim 15, wherein said additional anionic amphiphilic lipid is present in an amount ranging from 0.01 to 10% by weight with respect to the total weight of the composition.

17. A nanoemulsion according to claim 1, wherein said amount of oily phase ranges from 2 to 40% by weight with respect to the total weight of the composition.

18. A nanoemulsion according to claim 1, which has a turbidity ranging from 60 to 600 NTU.

19. A nanoemulsion according to claim 1, which is a cosmetic or dermatological composition.

20. A cosmetic or dermatological composition, comprising a nanoemulsion, wherein said nanoemulsion comprises oily phase globules dispersed in an aqueous phase, wherein said oily phase globules have an average size of less than 100 nm, and wherein said nanoemulsion further comprises:
(1) at least one amphiphilic lipid selected from the group consisting of nonionic amphiphilic lipids, anionic amphiphilic lipids, and mixtures thereof, wherein said anionic amphiphilic lipid or lipids are selected from the group consisting of (a) mixed esters of fatty acids and of fatty alcohols, of carboxylic acids and of glycerol; (b) alkyl ether citrates; (c) alkenyl succinates; (d) fatty esters of phosphoric acid; and (e) mixtures thereof; and
(2) at least one anionic polymer comprising at least one hydrophobic chain, and wherein the weight ratio of the amount of oily phase to the amount of amphiphilic lipid ranges from 1.2 to 10.

21. An ophthalmic vehicle, comprising a nanoemulsion according to claim 1.

22. A pharmaceutical composition, comprising a nanoemulsion according to claim 1.

23. A method for caring for, treating and/or making up the skin, comprising applying to skin a nanoemulsion, wherein said nanoemulsion comprises oily phase globules dispersed in an aqueous phase, wherein said oily phase globules have an average size of less than 100 nm, and wherein said nanoemulsion further comprises:
(1) at least one amphiphilic lipid selected from the group consisting of nonionic amphiphilic lipids, anionic amphiphilic lipids, and mixtures thereof, wherein said anionic amphiphilic lipid or lipids are selected from the group consisting of (a) mixed esters of fatty acids and of fatty alcohols, of carboxylic acids and of glycerol; (b) alkyl ether citrates; (c) alkenyl succinates; (d) fatty esters of phosphoric acid; and (e) mixtures thereof; and
(2) at least one anionic polymer comprising at least one hydrophobic chain, and wherein the weight ratio of the amount of oily phase to the amount of amphiphilic lipid ranges from 1.2 to 10.

24. A method for caring for and/or treating the hair, comprising applying to hair a nanoemulsion, wherein said nanoemulsion comprises oily phase globules dispersed in an aqueous phase, wherein said oily phase globules have an average size of less than 100 nm, and wherein said nanoemulsion further comprises:
(1) at least one amphiphilic lipid selected from the group consisting of nonionic amphiphilic lipids, anionic amphiphilic lipids, and mixtures thereof, wherein said anionic amphiphilic lipid or lipids are selected from the group consisting of (a) mixed esters of fatty acids and of fatty alcohols, of carboxylic acids and of glycerol; (b) alkyl ether citrates; (c) alkenyl succinates; (d) fatty esters of phosphoric acid; and (e) mixtures thereof; and
(2) at least one anionic polymer comprising at least one hydrophobic chain, and wherein the weight ratio of the amount of oily phase to the amount of amphiphilic lipid ranges from 1.2 to 10.

25. A method for caring for and/or moisturizing the skin, mucous membranes and/or scalp, comprising applying to skin, mucous membranes and/or scalp a nanoemulsion, wherein said nanoemulsion comprises oily phase globules dispersed in an aqueous phase, wherein said oily phase globules have an average size of less than 100 nm, and wherein said nanoemulsion further comprises:
(1) at least one amphiphilic lipid selected from the group consisting of nonionic amphiphilic lipids, anionic amphiphilic lipids, and mixtures thereof, wherein said anionic amphiphilic lipid or lipids are selected from the group consisting of (a) mixed esters of fatty acids and of fatty alcohols, of carboxylic acids and of glycerol; (b) alkyl ether citrates; (c) alkenyl succinates; (d) fatty esters of phosphoric acid; and (e) mixtures thereof; and
(2) at least one anionic polymer comprising at least one hydrophobic chain, and wherein the weight ratio of the amount of oily phase to the amount of amphiphilic lipid ranges from 1.2 to 10.

26. A process for thickening an oil-in-water nanoemulsion having oil globules with an average size of less than 100 nm, which consists in adding an anionic polymer comprising at least one hydrophobic chain to the said nanoemulsion.

27. A process for preparing a nanoemulsion, comprising:
(1) mixing an aqueous phase with and oily phase, with vigorous stirring, at a temperature of less than 45° C.;
(2) carrying out a stage of high-pressure homogenization at a pressure of greater than 5×10⁷ Pa; and
(3) adding at least one anionic polymer comprising at least one hydrophobic chain, to obtain said nanoemulsion.

28. A process according to claim 27, further comprising carrying out a subsequent high-pressure homogenization of said nanoemulsion at a pressure of greater than 5×10⁷ Pa.

29. A process according to claim 27, wherein said high-pressure homogenization is carried out at a pressure ranging from 6×10⁷ to 18×10⁷ Pa and a shear rate ranging from 2×10⁶ s⁻¹ to 5×10⁸ s⁻¹.
